(19) United States
(12) Patent Application Publication
(10) Pub. No.: US 2019/0274667 A1
(43) Pub. Date: Sep. 12, 2019

(54) ERGONOMIC HANDLE

(71) Applicants: Seth Freshly, Gahanna, OH (US); William Burgette, Dublin, OH (US)

(72) Inventors: Seth Freshly, Gahanna, OH (US); William Burgette, Dublin, OH (US)

(21) Appl. No.: 16/297,459

(22) Filed: Mar. 8, 2019

Related U.S. Application Data
(60) Provisional application No. 62/640,127, filed on Mar. 8, 2018.

(51) Int. Cl. A61B 17/00; B25G 1/10

(52) U.S. Cl. CPC A61B 17/00; A61B 17/3213; A61B 2017/00424; B25G 1/102

(57) ABSTRACT

A scalpel handle is uniquely shaped to provide gripping comfort while performing multiple styles of hand holds for long periods of time. There are textured surfaces to prevent slippage of fingers. The slope of the handle itself conforms to the natural curvature of the human hand. Embodiments achieve the above objectives by providing an ergonomic handle for precision surgical and dental use, among others. In some embodiments it is wider across the top surface as opposed to typical flat scalpels to provide a more efficient surface for applying pressure downwards during cutting procedures. It is also curved down and away from the blade/tool attachment area to conform to the pocket of the human hand. This ergonomically improved handle conforms to natural human hand shape, allows for better grip under stress, increased tactile feedback and cutting efficiency, and a comfortable grip during long term usage.

### CROSS-REFERENCE TO RELATED APPLICATIONS

This application claims the benefit of U.S. Provisional Patent Application No. 62/640,127, filed Mar. 8, 2018, which is/are all hereby incorporated by reference in their entirety.

### FIELD OF THE INVENTION

The present invention relates to handles for precision tools such as surgical and dental tools.

### BACKGROUND

Existing handles, particularly fine tool handles such as scalpels handles are traditionally flat and straight. A standard scalpel handle is flat, and the top ridge of the flat handle does not allow for an efficient surface for the fingers and thumbs to apply comfortable and appropriate pressure. The flat body handle does not provide proper gripping arrangements to prevent slippage of fingers.

### SUMMARY OF THE INVENTION

Embodiments relate to a handle for a user including: (a) a tool attachment; (b) a front part of the handle; (c) a middle part of the handle wherein the middle is about 0.75 inches wide immediately rear of (a) and narrows to about 0.5 inches wide at the rear of the middle part; and (d) a rear part of the handle which comprises a downward curving body which drops at least about 1.5 inches from the front portion of the rear part to the rear portion of the rear part. In some embodiments these are the only four parts and they directly connect to each other in the listed order. Embodiments also relate to a handle that (a) includes a top surface that slopes up from the portion near (a) to the portion near (c); wherein (a) includes a top surface textured to facilitate the user's hand grip; wherein (b) includes a top surface that is partially concave to facilitate the user's hand grip; wherein (b) includes a top surface textured to facilitate the user's hand grip; wherein (b) further comprises at least one side comprising a surface that comprises concave textured slopes; wherein (c) includes a hollow channel; wherein the at least one side facilitates index finger gripping with a palmar grip; wherein the top surface facilitates thumb gripping with a palm grip; and wherein the top surface facilitates index finger gripping with a pencil grip.

Embodiments relate to a handle for a user including: (a) a tool attachment; (b) a front part of the handle; (c) a middle part of the handle wherein the middle is about 10% of the user's hand length wide immediately rear of (a) and narrows to about 6.7% of the user's hand length wide at the rear of the middle part; and (d) a rear part of the handle which comprises a downward curving body which drops at least about 20% of the user's hand length from the front portion of the rear part to the rear portion of the rear part. In some embodiments these are the only four parts and they directly connect to each other in the listed order. Embodiments also relate to a handle that (a) includes a top surface that slopes up from the portion near (a) to the portion near (c); wherein (a) includes a top surface textured to facilitate the user's hand grip; wherein (b) includes a top surface that is partially concave to facilitate the user's hand grip; wherein (b) includes a top surface textured to facilitate the user's hand grip; wherein (b) further comprises at least one side comprising a surface that comprises concave textured slopes; wherein (c) includes a hollow channel; wherein the at least one side facilitates index finger gripping with a palmar grip; wherein the top surface facilitates thumb gripping with a palm grip; and wherein the top surface facilitates index finger gripping with a pencil grip.

### BRIEF DESCRIPTION OF THE DRAWINGS

FIG. 1 illustrates two standard scalpel handles in the current art.

FIG. 2 is a left side view of the handle of the current invention.

FIG. 3 is a right side view of the handle of the current invention.

FIG. 4 is a bottom side view of the handle of the current invention.

FIG. 5 illustrates a palm grip used with the current invention.

FIG. 6 illustrates a pencil grip or tripod grip used with the current invention.

FIG. 7 illustrates a palmar grip used with the current invention.

### DETAILED DESCRIPTION

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

One objective of the disclosed embodiments provides a scalpel handle which is uniquely shaped to provide gripping comfort while performing multiple styles of hand holds for long periods of time. There are textured surfaces to prevent slippage of fingers. The slope of the handle itself conforms to the natural curvature of the human hand.

A further objective is to avoid designing the ergonomics with only one size/shape of hand in mind. The rest of the handle provides no additional finger indentations as hand sizes are drastically different and these would add pressure points when holding the device in various fashions.

An embodiment achieves the above objectives by providing an ergonomic handle for precision surgical and dental use, among others. In some embodiments it is wider across the top surface as opposed to typical flat scalpels to provide a more efficient surface for applying pressure downwards during cutting procedures. It is also curved down and away from the blade/tool attachment area to conform to the pocket of the human hand.

This ergonomically improved handle conforms to natural human hand shape, allows for better grip under stress, increased tactile feedback and cutting efficiency, and a comfortable grip during long term usage. Thus, the handle is ergonomic and efficient.

On embodiment of the handle has a downward curving extended body which drops over about 1.5 inches (“in”) from the front of the handle to the back portion of the handle. The width of the handle is about 0.75 in across the top textured surface behind the blade/tool attachment and then curving down and away from the blade/tool attachment with a decreasing width by symmetrical concaving textured slopes on each side to a width of 0.50 in extending to the end of the handle. An upward sloping textured surface immediately behind the blade/tool attachment extends up and then back down a textured surface leading away from the blade/tool attachment. Overall length is 5.0 in from the front to the back of the handle. With regard to the following handle embodiment, which was also described above, note that the parts a, b, c, and d typically will be attached to each other in the listed order, but other embodiments are possible, such as intervening parts: (a) a tool attachment; (b) a front part of the handle; (c) a middle part of the handle wherein the middle is about 10% of the user's hand length wide immediately rear of (a) and narrows to about 6.7% of the user's hand length wide at the rear of the middle part; and (d) a rear part of the handle which comprises a downward curving body which drops at least about 20% of the user's hand length from the front portion of the rear part to the rear portion of the rear part. In some embodiments these are the only four parts and they directly connect to each other in the listed order.

These measurements are only one embodiment, and other measurements are contemplated. In addition, the above measurements may vary, and any such stated measurement should be interpreted as “about” or “approximately”, and a certain tolerance of 20% or more is contemplated. So, for instance, 0.75 in measurement as stated above could be 0.6-0.9 in. While the invention is generally useful for many sized hands, other sizes can be made for different sized user. For instance, it is known in the art that the length of the average human male hand is about 7.5 in, so the 0.75 in length as cited above is about 10% of that hand length value. For instance, a hand that is 6.75 in long (90% of 7.5 in) the embodiment might have all the above values multiplied by 90%. Note that 6.75 in is about the length of the average human female hand.

Note that the tool attachment typically is selected or constructed from attachment standards that exist in the art (as well as novel ones) selected or constructed for the intended purpose. If a scalpel blade is to be attached, there exists a standard scalpel blade attachment, typically referred to as a lock and key device, which will be part (a) of the handle as described above. Other standard attachment tools, as well as non-standard attachment tools, are contemplated.

Embodiments herein provide a handle which is uniquely shaped to provide gripping comfort while performing multiple styles of hand holds for long periods of time. There are textured surfaces to prevent slippage of fingers. The slope of the handle itself conforms to the natural curvature of the human hand. A further objective is to avoid designing the ergonomics with only one size/shape of hand in mind. The rest of the handle provides no additional finger indentations as hand sizes are drastically different and these would add pressure points when holding the device in various fashions. The present invention achieves the above objectives by providing an ergonomic handle for precision, surgical, and dental use. It is wider across the top surface as opposed to typical flat scalpels to provide a more efficient surface for applying pressure downwards during cutting procedures. It is also curved down and away from the blade/tool attachment area to conform to the pocket of the human hand.

Multiple tool types can be used with the handle. Dental, hobby, and other medical tools can used with the disclosed handle. A surgical blade may be attached. The handle can also be used with many other attachments, such as a surgical tool, electro-surgical tool, microsurgery tool, dental tool, engraving tool, soldering tool, artwork cutting tool or any other hand held tool, particularly for fine motor control work.

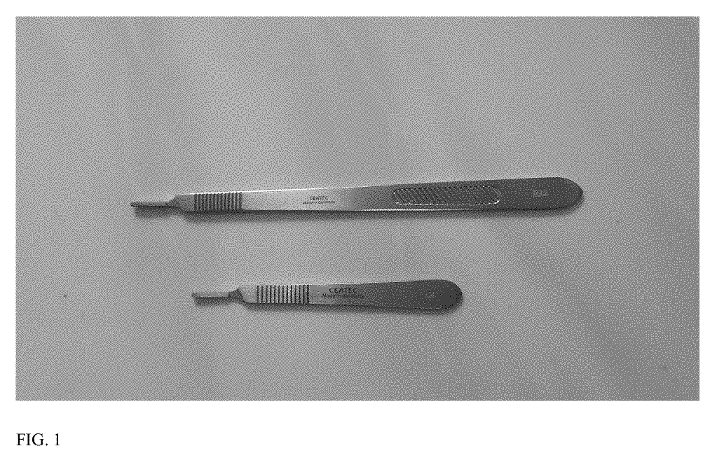

FIG. 1 illustrates two standard scalpel handles in the current art.

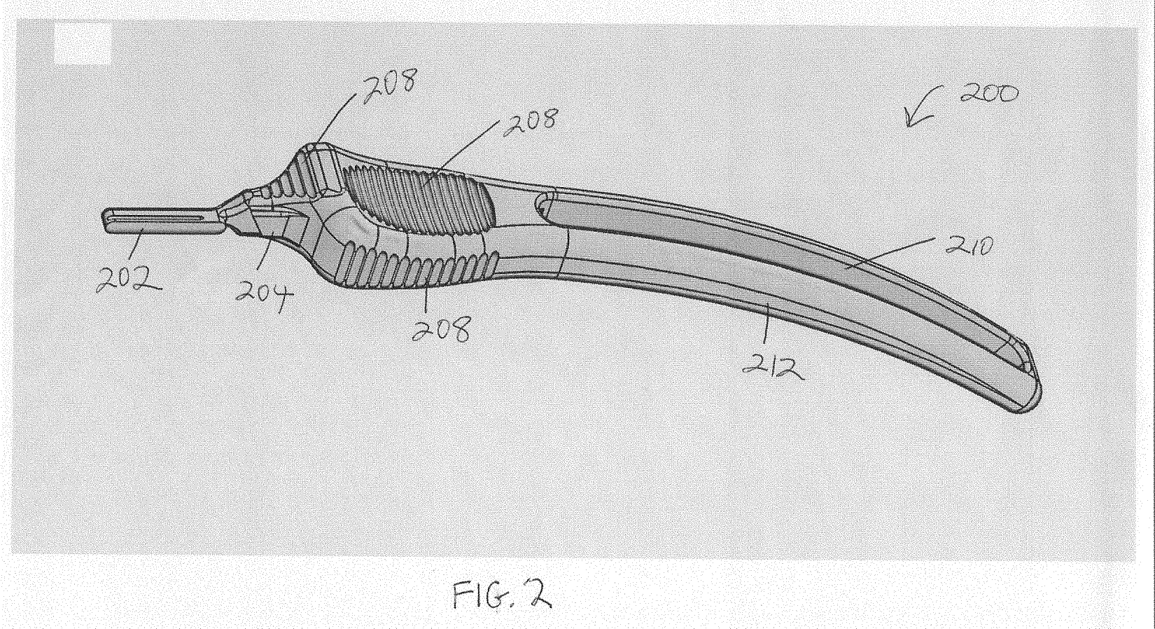

FIG. 2 is a left side view of the handle of the current invention 200. The illustration includes a tool attachment portion 202, a front portion 204, typically narrow, a middle portion 206, in one embodiment about 0.75 in wide at the front and 0.5 in wide at the back, and a rear portion 212 which in one embodiment drop at least about 1.5 in and has a hollow channel 210. In some embodiments the hollow channel has an open surface, in others it does not. The hollow channel is intended to reduce the weight of the handle, though other means for reducing the weight can also be used. Embodiments of the handle have textured surfaces 208 as shown, and as described above.

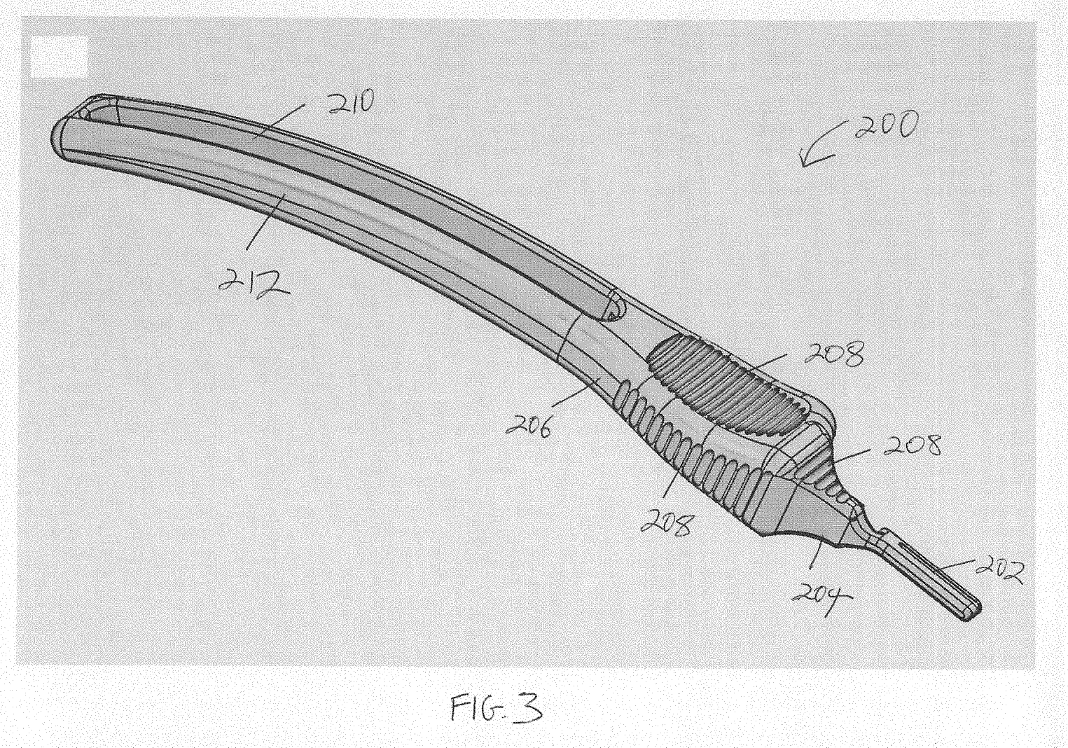

FIG. 3 is a left side view of the handle of the current invention with the same elements (though not necessarily all of them can be seen from this view).

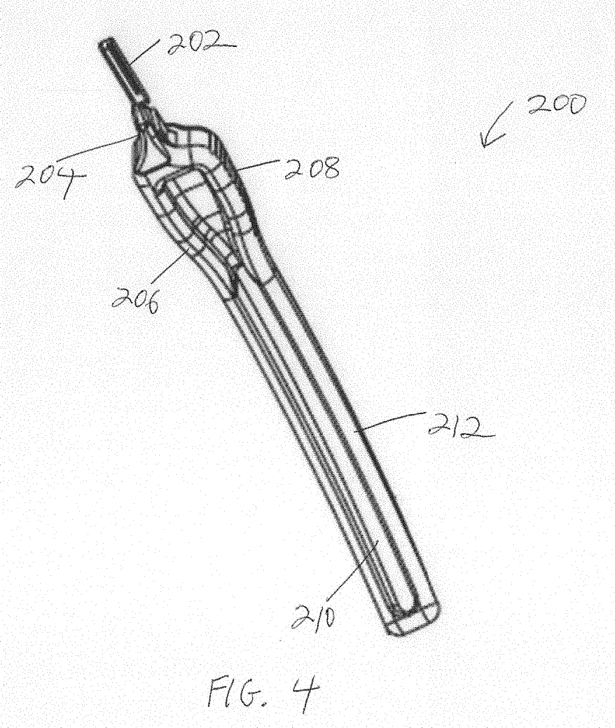

FIG. 4 is a right side view of the handle of the current invention with the same elements (though not necessarily all of them can be seen from this view).

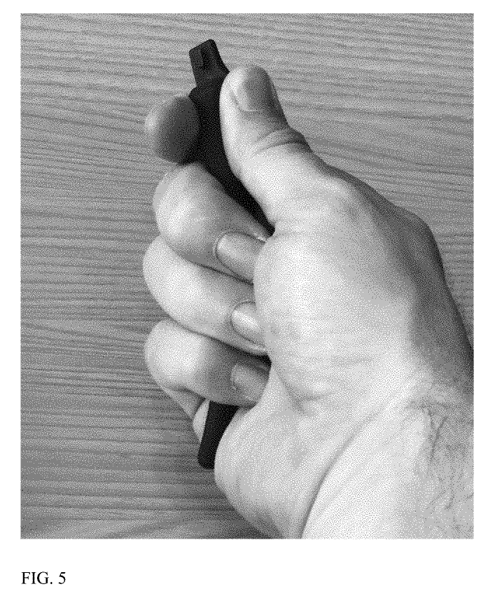

FIG. 5 illustrates a palm grip used with the current invention. As is well known in the art, the palm grip involves holding the handle within the palm of the hand. The thumb can be placed on the upper surface of the handle to create more force and increase control. It is best suited for long incisions requiring greater pressure; due to the position of the blade on the skin, it results in better contact between the blade and the tissues compared to the pencil grip. This grip is rarely used to create incisions but can be used with other instruments such as periosteal elevators.

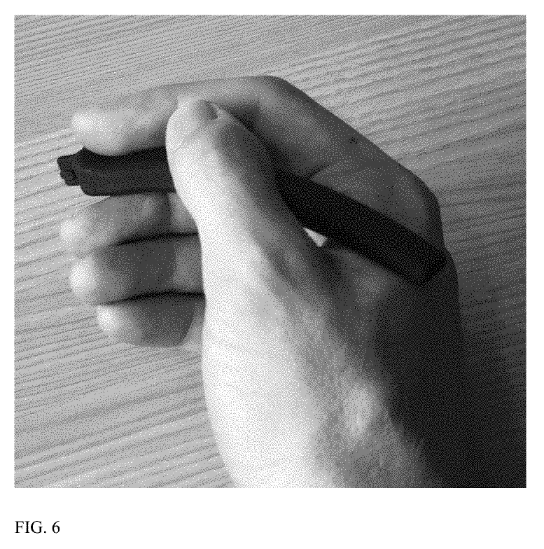

FIG. 6 illustrates a pencil grip used with the current invention. This is also called the tripod grip. As is well known in the art, with this grip the handle is held with the tips of the first and second fingers and the tip of the thumb with the handle resting on the fleshy base of the index finger and thumb. Care should be taken not to allow the handle to rest too far along the index finger as this promotes an unstable grip and cramped fingers.

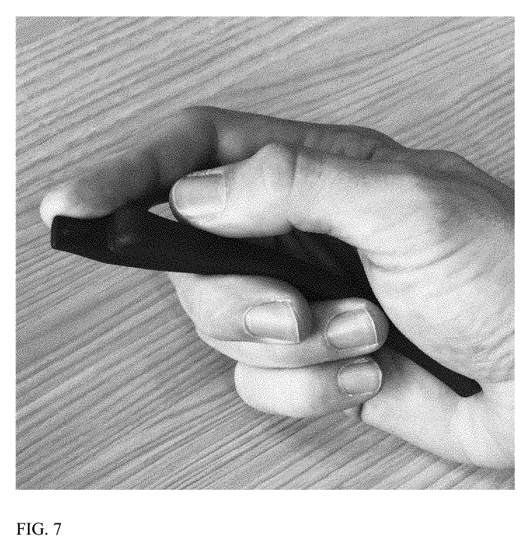

FIG. 7 illustrates a palmar grip used with the current invention. This is also called the dinner knife grip. Also called the “dinner knife” grip. As is well known in the art, with this grip handle is held with the second through fourth fingers and secured along the base of the thumb, with the index finger extended along the top rear of the blade and the thumb along the side of the handle. This grip is best for initial incisions and larger cuts.

### Terminology

The terminology used herein is for the purpose of describing particular aspects only and is not intended to be limiting of the disclosure. As used herein, the singular forms “a,” “an,” and “the” are intended to comprise the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. In this specification, reference is made in detail to specific embodiments of the invention. Some of the embodiments or their aspects are illustrated in the drawings.

For clarity in explanation, the invention has been described with reference to specific embodiments, however it should be understood that the invention is not limited to the described embodiments. On the contrary, the invention covers alternatives, modifications, and equivalents as may be included within its scope as defined by any patent claims. The following embodiments of the invention are set forth without any loss of generality to, and without imposing limitations on, the claimed invention. In the following description, specific details are set forth in order to provide a thorough understanding of the present invention. The present invention may be practiced without some or all of these specific details. In addition, well known features may not have been described in detail to avoid unnecessarily obscuring the invention.

In addition, it should be understood that steps of the exemplary methods set forth in this exemplary patent can be performed in different orders than the order presented in this specification. Furthermore, some steps of the exemplary methods may be performed in parallel rather than being performed sequentially.

While various embodiments constructed according to the principles disclosed herein, as well as specific components, have been described above, it should be understood that they have been presented by way of example only, and not limitation. Thus, the breadth and scope of the invention should not be limited by any of the above-described exemplary embodiments, but should be defined only in accordance with the following claims and their equivalents.

1. A handle for a user comprising:
(a) a tool attachment;
(b) a front part of the handle;
(c) a middle part of the handle, wherein the middle is about 0.75 inches wide immediately rear of (a) and narrows to about 0.5 inches wide at the rear of the middle part; and
(d) a rear part of the handle which comprises a downward down curving body which drops at least about 1.5 inches from the front portion of the rear part to the rear portion of the rear part.

2. The handle of claim 1 wherein (b) further comprises a top surface that slopes up from the portion near (a) to the portion near (c).

3. The handle of claim 1 wherein (b) further comprises a top surface textured to facilitate the user's hand grip.

4. The handle of claim 1 wherein (c) further comprises a top surface that is partially concave to facilitate the user's hand grip.

5. The handle of claim 1 wherein (c) further comprises a top surface textured to facilitate the user's hand grip.

6. The handle of claim 1 wherein (c) further comprises at least one side comprising a surface that comprises concave textured slopes.

7. The handle of claim 1 wherein (d) further comprises a hollow channel.

8. The handle of claim 6 wherein the at least one side facilitates index finger gripping with a palmar grip.

9. The handle of claim 3 wherein the top surface facilitates thumb gripping with a palm grip.

10. The handle of claim 3 wherein the top surface facilitates index finger gripping with a pencil grip.

11. A handle for a user comprising:
(a) a tool attachment;
(b) a front part of the handle;
(c) a middle part of the handle, wherein the middle is about 10% of the user's hand length wide immediately rear of (a) and narrows to about 6.7% of the user's hand length wide at the rear of the middle part; and
(d) a rear part of the handle which comprises a downward down curving body which drops at least about 20% of the user's hand length wide from the front portion of the rear part to the rear portion of the rear part.

12. The handle of claim 11 wherein (b) further comprises a top surface that slopes up from the portion near (a) to the portion near (c).

13. The handle of claim 11 wherein (b) further comprises a top surface textured to facilitate the user's hand grip.

14. The handle of claim 11 wherein (c) further comprises a top surface that is partially concave to facilitate the user's hand grip.

15. The handle of claim 11 wherein (c) further comprises a top surface textured to facilitate the user's hand grip.

16. The handle of claim 11 wherein (c) further comprises at least one side comprising a surface that comprises concave textured slopes.

17. The handle of claim 11 wherein (d) further comprises a hollow channel.

18. The handle of claim 16 wherein the at least one side facilitates index finger gripping with a palmar grip.

19. The handle of claim 13 wherein the top surface facilitates thumb gripping with a palm grip.

20. The handle of claim 13 wherein the top surface facilitates index finger gripping with a pencil grip.
